(19) United States
(12) Patent Application Publication
(10) Pub. No.: US 2021/0186373 A1
(43) Pub. Date: Jun. 24, 2021

(54) Attachment Device and Method for a Sample Collection Device for Obtaining Samples from Respiratory Air

(71) Applicant: Robert Bosch GmbH, Stuttgart (DE)

(72) Inventors: Michael Stumber, Korntal-Muenchingen (DE); Veronika Schleper, Leinfelden-Echterdingen (DE); Philipp Jung, Pfinztal (DE); Katrin Luckert, Leonberg (DE)

(21) Appl. No.: 16/756,394

(22) PCT Filed: Oct. 16, 2018
(86) PCT No.: PCT/EP2018/078168

§ 371 Date: Apr. 15, 2020

(30) Foreign Application Priority Data
Oct. 26, 2017 (DE) 10 2017 219 180.5

(51) Int. Cl. A61B 5/097; A61B 5/08; A61B 10/00

(52) U.S. Cl. CPC A61B 5/097; A61B 5/082; A61B 2010/0087; A61B 2560/0443; A61B 10/00

(57) ABSTRACT

An attachment device for a sample collection device for obtaining samples from exhaled respiratory air includes an inlet tube, a dip tube for discharging the respiratory air from the sample collection device, and at least a portion of a spiral path. The portion runs into an outlet opening which is designed to conduct the respiratory air to the sample collection device.

### PRIOR ART

The invention proceeds from a device or a method of the type according to the independent claims.

Samples of the air exhaled by a human can be collected for analysis. It is possible to examine both the gaseous fraction and also the aerosols, originating from the lungs, and the breath condensate.

Various apparatuses for obtaining respiratory samples are described in the literature, ranging from collection of gas samples, collection of various “fractions” (from defined regions of the lungs or of the airways), through collection of liquid, in most cases condensate from the saturated exhaled respiratory air, to tests for capturing defined substances directly in the respiratory air or for detecting them by sensors (the most popular example being “FeNO”—fractional exhaled nitric oxide).

### DISCLOSURE OF THE INVENTION

Against this background, the approach presented here concerns an attachment device for a sample collection device for obtaining samples from exhaled respiratory air, and also a method for obtaining samples from exhaled respiratory air, according to the main claims.

Advantageous developments and improvements of the device set out in the independent claim are possible by means of the measures set out in the dependent claims.

An attachment device for a sample collection device serves to ensure that aerosols present in the respiratory air are collected in a simplified and targeted manner, which aerosols, for example as carriers of proteins and biomarkers, allow strong conclusions to be drawn regarding the state of health of the person breathing.

An attachment device for a sample collection device for obtaining samples from exhaled respiratory air is proposed, wherein the attachment device comprises an inlet tube, a dip tube for discharging the respiratory air from the sample collection device, and at least a portion of a spiral path, wherein the portion runs into an outlet opening which is designed to conduct the respiratory air to the sample collection device.

An attachment device can be an add-on for a sample collection device, wherein the attachment device in conjunction with the sample collection device forms a centrifugal separator. A sample collection device can be a reaction vessel for small to medium sample volumes in the microliter to milliliter range. The spiral path, and thus the portion of the spiral path, can be part of a spiral. The outlet opening can form an admission cross section for the flow of the respiratory air from the spiral path into the sample collection device. The outlet opening for conducting the respiratory air into the sample collection device can be formed in a wall portion of the attachment device arranged, in the assembled state of the sample collection device, in the region of an opening of the sample collection device. The respiratory air from the spiral path can thus flow through the outlet opening into the sample collection device. The spiral path can form a flow channel for the respiratory air. For example, an opening angle of the outlet opening of the spiral path can be between 10° and 180°. The spiral path can extend helically in the attachment device.

The attachment device can be used to obtain aerosols or condensate from respiratory samples. The aerosols contained in the exhaled air originate from the film of epithelial liquid lining the lungs and serve as carriers of physiological substances secreted by the lungs, often designated as “biomarkers”. In contrast to a sample generated by a cooling device, in which dilution would take place, the samples obtained contain a higher concentration of biomarkers, which extends the range of possible analysis methods and makes analysis easier. The attachment device is consequently adapted to capture the respiratory aerosols, which have an average diameter of 300 nm according to current scientific knowledge, and thus to accumulate the biomarkers contained in the aerosols. Control of the respiratory process is also possible, and it is advantageous for the desired reproducibility and standardization. Advantageously, the sampling does not require any manual operation of the equipment by an operator. In the approach described here, the collecting can in principle also be carried out without cooling or condensation of the exhaled air. However, it may be advantageous for the sample collection device or parts thereof to be temperature controlled, in order to prevent renewed evaporation of the sample or parts thereof or in order to better preserve the biomarkers. Suitable temperatures for the temperature control of the sample are in the range of between >0° C. and <30° C. at the sample site.

According to one embodiment, the inlet tube can have a free end designed as a mouthpiece or can be connected to a mouthpiece or designed as a mouthpiece. For this purpose, the inlet tube can have an interface for fastening the mouthpiece. Such an interface can permit a form-fit or force-fit connection between the mouthpiece and the attachment device. For example, the interface can comprise a thread or a clip element. The mouthpiece can thus be integrated or can be designed as an extra plug-on part. The inlet tube can thus have a connection to the mouthpiece to permit admission of the sample. The inlet tube can be designed to allow the respiratory air to flow into the spiral path. The spiral path here serves to control the direction of flow of the respiratory air in a targeted manner, in order to be able to collect a large proportion of the aerosols contained in the respiratory air and carrying the released biomarkers. A user can blow respiratory air into the mouthpiece.

Furthermore, the attachment device has a dip tube. The dip tube serves to discharge the respiratory air from the sample collection device. Continuous use is thus possible.

According to one embodiment, the dip tube can have a protrusion which protrudes beyond a stop of the attachment device for the sample collection device. Protrusion of the dip tube into the sample collection device supports the formation of a stable cyclonic flow of the respiratory air inside the sample collection device. The stop can be arranged on a side of the attachment device lying opposite a free end of the dip tube. The stop can be designed to limit the entry of the sample collection device into the attachment device. For example, the sample collection device can be screwed into the attachment device as far as the stop. The respiratory air freed of the sample can escape from the attachment device via the free end of the dip tube.

Longitudinal axes of the dip tube and of the inlet tube can be at an acute angle to each other. Here, for example, a pump can be attached via a hose, which pump assists the exhalation process by reducing the respiratory resistance felt by the user.

The respiratory process is advantageously performed by taking regular but at the same time deep breaths at rest. A 10 minute sample collection time with respiration at rest is recommended.

According to one embodiment, the attachment device can have has an interface for mechanical, reversible connection of the attachment device and the sample collection device. The connection can thus also be released again. The mechanical connection of the attachment device to the sample collection device can be effected via a force-fit screw thread or a form-fit plug connection. The mechanical connection can be designed to be releasable such that, after the sample has been collected, the sample collection device can be released from the attachment device and, for example, plugged into laboratory equipment.

A collector is proposed having an attachment device and having a sample collection device mechanically coupled to the attachment device. Such a collector can be a unit that is easily manageable by a user. Thus, in one illustrative embodiment, the sample collection device can be designed as a reaction vessel with a volume of between 0.05 ml and 50 ml.

According to one embodiment, the attachment device can taper at a side directed toward the sample collection device and can have an opening. The attachment device can be placed or placeable directly onto the sample collection device and can be suitable for further processing steps in the laboratory. The side of the attachment device directed toward the sample collection device is typically the underside during use of the collector. The opening of the attachment device can be arranged at an end portion of the taper and can constitute a passage to the sample collection device. The attachment device can be advantageously constructed such that it is placed directly onto the sample collection device. This construction is suitable for collecting larger sample quantities, without adversely affecting the flow conditions during collection.

The sample collection device can thus be designed as a commercially available reaction vessel. The reaction vessel can have a volume of between 0.05 ml and 3 ml, for example. The sample collection device can also be designed as a centrifuge tube with a volume of between 2.5 ml and 25 ml, for example with a volume of 15 ml.

A method for obtaining samples from exhaled air is proposed, wherein the method comprises the following steps:

guiding the exhaled air into a centrifugal separator; and

collecting the samples from the exhaled air at the wall of the centrifugal separator using the centrifugal force.

Advantageously, the guiding of the exhaled air into the centrifugal separator can be assisted by a pump. The method can comprise a step of separation, in which a sample collection vessel of the centrifugal separator is separated from an attachment device of the centrifugal separator.

According to one embodiment, a centrifugal separator can be used for the purpose of obtaining samples from exhaled air. Using the spiral, which controls a flow of the respiratory air, and using the centrifugal force, the centrifugal separator permits effective collection of the aerosols contained in the breath, and of the biomarkers contained in the aerosols of the respiratory air samples for diagnostic purposes.

Illustrative embodiments of the approach presented here are shown in the drawings and are explained in more detail in the description below. In the drawings:

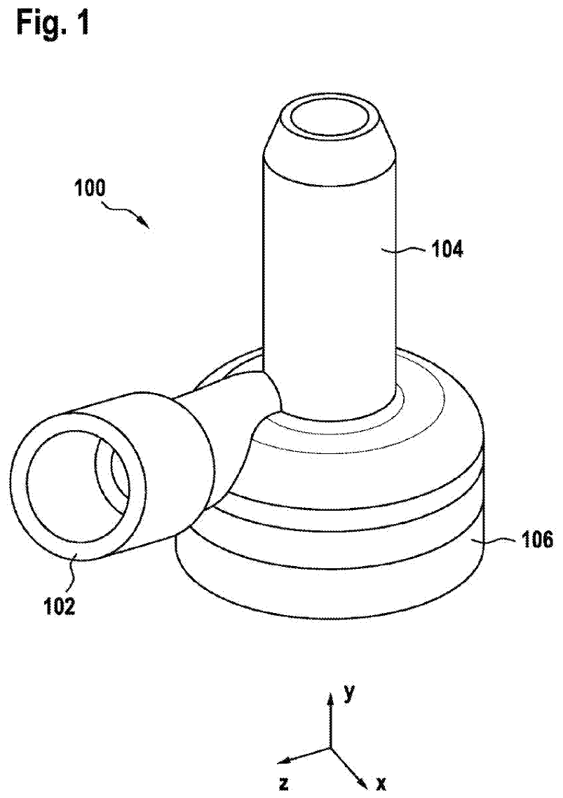

FIG. 1 shows a view of an attachment device according to one illustrative embodiment;

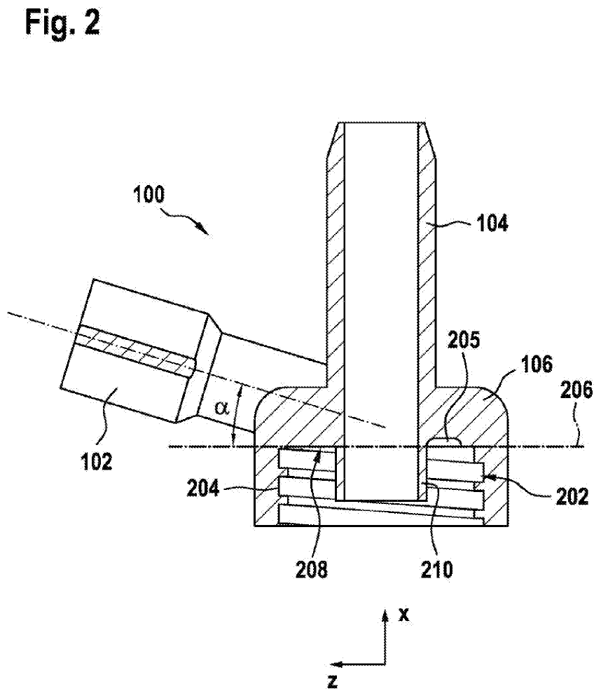

FIG. 2 shows a cross-sectional view of an attachment device according to one illustrative embodiment;

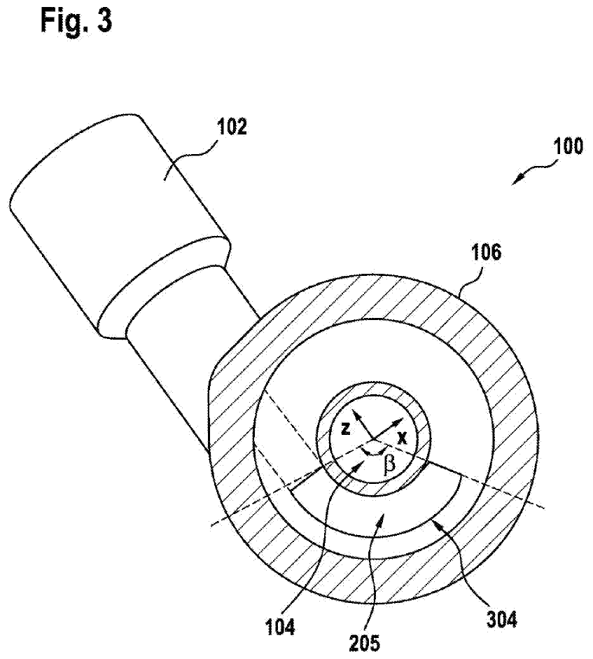

FIG. 3 shows a view of an attachment device according to one illustrative embodiment;

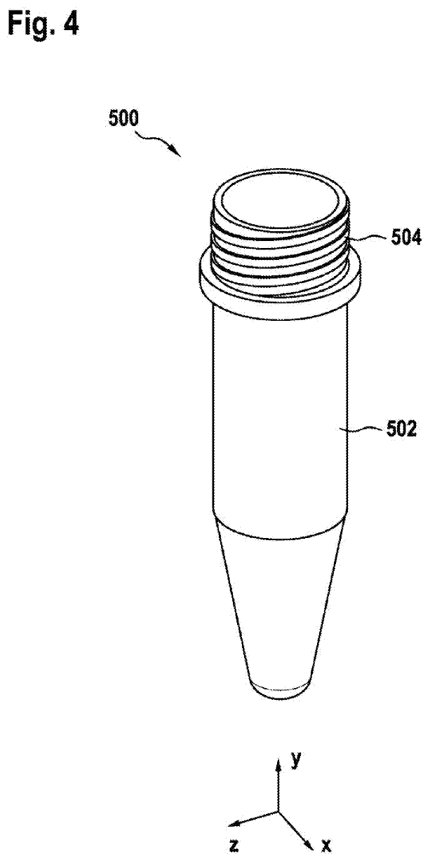

FIG. 4 shows a schematic view of a sample collection device according to one illustrative embodiment;

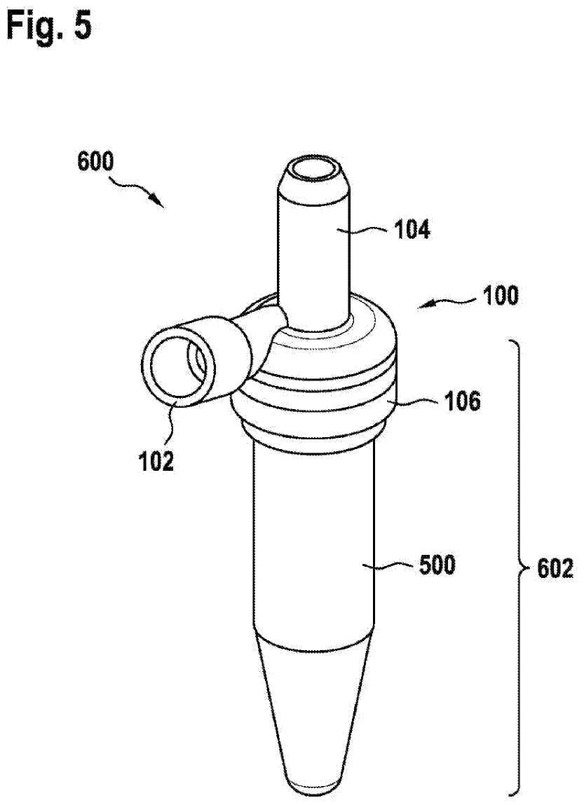

FIG. 5 shows a view of a collector according to one illustrative embodiment;

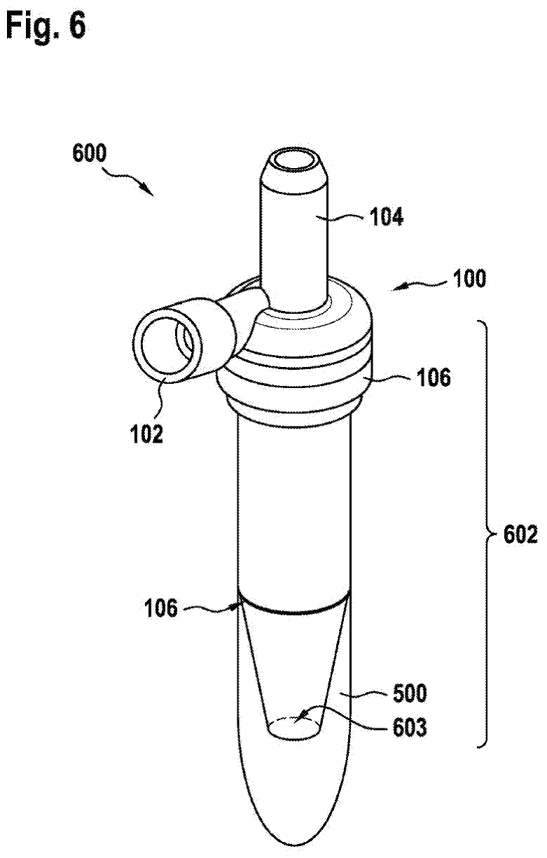

FIG. 6 shows a view of a collector according to a further illustrative embodiment; and

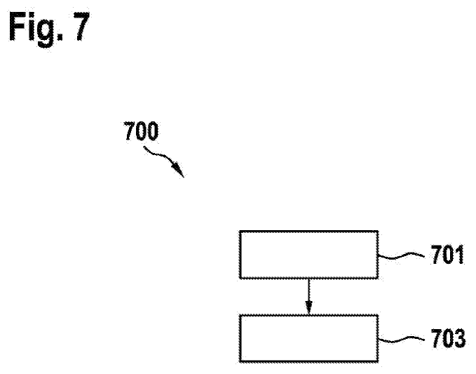

FIG. 7 shows a flow chart of an illustrative embodiment of a method for obtaining samples from exhaled air according to an illustrative embodiment.

In the following description of expedient illustrative embodiments of the present invention, the elements shown in the various figures and having similar effects are designated by the same or similar reference signs, thereby avoiding repeated description of these elements.

FIG. 1 shows a view of an attachment device 100 according to one illustrative embodiment. The attachment device 100 comprises an inlet tube 102 and a dip tube 104. The attachment device 100 serves as an attachment for a sample collection device shown in FIG. 5, wherein the attachment device 100, in conjunction with the sample collection device, constitutes a collector in the form of a centrifugal separator, as is shown in FIG. 6.

The attachment device 100 is provided for obtaining samples from the air exhaled by a living being. For this purpose, a free end of the inlet tube 102 according to one illustrative embodiment is designed as a mouthpiece or is designed to receive such a mouthpiece.

The inward flow of respiratory air takes place through the inlet tube 102, which is mounted eccentrically on a cylindrical main body 106 of the attachment device 100. For this purpose, a person places the ergonomically shaped mouthpiece of the inlet tube 102 into his mouth and breathes several times into the inlet tube 102. The respiratory process is advantageously performed by taking regular but at the same time deep breaths at rest. The air leaves the attachment device 100 via the dip tube 104, after the exhaled air has circulated in order to deposit a sample in the sample collection device. According to this illustrative embodiment, the dip tube 104 is routed centrally through the main body 106. With the sample collection device assembled, the dip tube 104 can protrude into the sample collection device. According to one illustrative embodiment, longitudinal axes of the inlet tube 102 and of the dip tube 104 are at an acute angle to each other.

According to one illustrative embodiment, the attachment device 100 serves to allow simple and targeted collection of aerosols that are released from the film of epithelial liquid lining the lungs and that pass into the breath. In each breathing process, aerosols are generated and are exhaled in a small concentration as droplets with a diameter of 200-1000 nm. Aerosols serve as carriers of proteins and biomarkers, which provide a revealing and valuable picture of the state of health of the lungs and of the person breathing. The task of the attachment device is therefore to collect what are known as exhaled breath aerosols and condensate (EBAC). The attachment device serves to permit reproducible and standardized sampling of EBAC which, by means of an exhaled breath and aerosol collector, traps a sufficiently large quantity of aerosols, thus making available a sufficiently high concentration of biomarkers for analysis of the respiratory air. The collecting of aerosols is thus addressed in a targeted manner. A further aim of this approach is to collect aerosols as close as possible to existing laboratory systems such as reaction vessels, to permit a use that is as versatile as possible, and to reduce the use of special accessories.

A y axis of the attachment device 100 corresponds to a longitudinal axis of the dip tube 104. An x-z plane of the attachment device 100 is perpendicular to the y axis.

FIG. 2 shows a longitudinal sectional view of an attachment device 100 according to one illustrative embodiment. The attachment device 100 shown in FIG. 2 can be, for example, the attachment device 100 shown in FIG. 1. In addition to the inlet tube 102 and the dip tube 104, the main body 106 of the attachment device 100 comprises an interface 202, wherein the interface 202 serves to connect the attachment device 100 to a thread 204 of the sample collection device, and an outlet opening 205, which conducts the respiratory air to the sample collection device.

FIG. 2 also shows a sectional plane 206. The inlet tube 102, which is mounted eccentrically on the attachment device 100, lies axially at an angle α of 0° to 70°, advantageously of 0° to 140°, to said sectional plane 206. A longitudinal axis of the dip tube 104, which discharges the respiratory air from the sample collection device, corresponds to a longitudinal axis of a cylindrical portion of the main body 106 having the thread 204 and the y axis of the attachment device 100. The thread 204 is designed as an internal thread. The thread 204 is routed as far as a stop 208 of the main body 106. When the sample collection device, which has an external thread corresponding to the thread 204, is screwed completely into the thread 204, an end portion of the wall of the sample collection device bears on the stop 208. By way of the thread 204, the sample collection device can be mounted on the attachment device 100 so as to be able to be released again from the latter. The dip tube 104 has a protrusion 210 which extends beyond the stop 208 and thus extends into the sample collection device when the sample collection device is fitted. The stop 208 is designed about a circumference. According to this illustrative embodiment, the outlet opening 205 adjoins an outer wall of the protrusion 210 of the dip tube 104 but is spaced apart from the thread 204, since the vessel wall of the sample collection device, also designated as collection vessel, is still between them. This is configured such that there is no step between the outlet opening and the collection vessel. Thus, the stop 208 can form a closed ring. The outlet opening 205 is formed in a cover portion of the main body 106 spanning the thread 204. The inlet tube 102 is connected to the main body 106 at the cover portion. Arranged inside the cover portion is the spiral path via which the respiratory air is guided from the inlet tube 102 to the outlet opening 205.

FIG. 3 shows a view of the attachment device 100 according to one illustrative embodiment. The attachment device 100 shown in FIG. 3 can be, for example, the attachment device 100 shown in FIG. 1 and FIG. 2. The cross section of the view of the attachment device 100 is along the sectional plane 206 shown in FIG. 2.

The attachment device 100 comprises the inlet tube 102, the dip tube 104 and at least one portion of a spiral, here a spiral path 304. The spiral path 304 extends over the outlet opening 205, wherein the outlet opening 205 is designed to conduct the respiratory air to the sample collection device. According to one illustrative embodiment, the outlet opening 205 is designed as a segment of a circle.

The inlet tube 102 is designed to allow the respiratory air to flow tangentially into the spiral path 304. According to one illustrative embodiment, the spiral path 304 has a rotation angle of 100°. The respiratory air flowing in is set in a circular path by the spiral path 304, wherein, by means of an optional tapering of the conically shaped sample collection device adjoining the attachment device 100, a speed of rotation of the respiratory air increases, such that the aerosols of the respiratory air are flung onto the wall of the sample collection device by the centrifugal force and are decelerated to the extent that they detach from the respiratory air and settle in the lower part of the sample collection device.

According to this illustrative embodiment, the outlet opening 205 is designed as a segment of a circle. The outlet opening 205 is arranged outside the dip tube 104. An opening angle β of the outlet opening 205 generally lies between a value of 10° and 180° and moreover depends on a pitch parameter of the spiral.

FIG. 4 shows a schematic view of a sample collection device 500 according to one illustrative embodiment. The sample collection device 500 comprises a conically shaped collection body 502 for collecting the aerosols from the respiratory air, and a screw thread 504 for screwing the sample collection device 500 into the thread of the attachment device.

The mechanical connection of the attachment device to the sample collection device 500 can be effected via the screw thread 504 or alternatively, for example, via a plug connection. The mechanical connection is releasable according to one illustrative embodiment, such that the sample collection device 500 can be released from the attachment device after the sample of respiratory air has been collected. According to one illustrative embodiment, the sample collection device 500 is designed to be attached directly to laboratory equipment without the need to transfer the sample of respiratory air to another vessel.

FIG. 5 shows a collector 600 according to one illustrative embodiment. The collector 600 comprises the attachment device 100 and also the sample collection device 500 coupled mechanically to the attachment device 100. According to one illustrative embodiment, the attachment device 100 can be secured to the sample collection device 500 using a plug connection or a thread, possibly with the aid of an additional seal.

The attachment device 100 is connected via the screw thread to the sample collection device 500, which holds 1.5 ml for example. The geometric dimensions and a volumetric flow rate of 18 l/min result in a speed of admission of the respiratory air of ca. 80 m/s at a pressure drop of ca. 50 mbar. The full flow of the respiratory air in the sample collection device 500 has a maximum speed of ca. 40 m/s. The calculated limit grain diameter is 250 nm.

The mechanical connection of the attachment device 100 to the sample collection device 500 results in a cyclonic centrifugal separator 602, wherein the centrifugal separator 602 is used for the purpose of obtaining samples from the exhaled respiratory air. The respiratory air is caused to flow in rotation with the aid of the centrifugal separator 602, such that the aerosols contained in the respiratory air are deposited and run off the wall of the sample collection device 500. The deposition of the aerosols from the respiratory air is effected by utilization of the centrifugal force, for example with low radii of curvature, high speeds and small cross sections.

The attachment device 100 is constructed such that it is placed directly onto the sample collection device 500, wherein the sample collection device 500 is suitable for the further processing steps in the laboratory. Before they are processed, the collected samples do not have to be transferred to another vessel, as is necessary in the currently available appliances. In the present design example of the collector 600, the maximum degree of efficacy of the centrifugal separator 602 is reached starting from a volumetric flow rate of ca. 18 l/min. This range is achievable during the maneuver of human respiration. The flow of the respiratory air deposits more than half of all the particles with a diameter of 400 nm or more in the sample collection device 500. For lower volumetric flow rates, the speed of admission can be adapted for example by a smaller cross section in the inlet, in order to maximize the degree of efficacy of the centrifugal separator.

FIG. 6 shows a further collector 600 according to one illustrative embodiment. The collector 600 comprises the attachment device 100 and also the sample collection device 500 coupled mechanically to the attachment device 100. According to one illustrative embodiment, the attachment device 100 can be secured to the sample collection device 500 using a plug connection or a thread, possibly with the aid of an additional seal.

The mechanical connection of the attachment device 100 to the sample collection device 500 results in a cyclonic centrifugal separator 602, wherein the centrifugal separator 602 is used for the purpose of obtaining samples from the exhaled respiratory air. The respiratory air is caused to flow in rotation with the aid of the centrifugal separator 602, such that the aerosols contained in the respiratory air are deposited and run off the wall of an extended attachment device 100. The deposition of the aerosols from the respiratory air is effected by utilization of the centrifugal force, for example with low radii of curvature, high speeds and small cross sections. The centrifugal separator has an opening 603 on its underside. At its lower end, the centrifugal separator has a tapering portion which runs into the opening 603. The collected aerosols are driven downward by the air stream in the cyclone and reach the sample collection vessel 500 lying below. Compared to the design in FIG. 5, this has the advantage that the geometry of the cyclone and therefore the air stream do not increase by the deposition of a sample quantity at the bottom of the sample collection vessel. This design is therefore also suitable for the collection of larger sample quantities.

The attachment device 100 is constructed such that it is placed directly onto the sample collection device 500, wherein the sample collection device 500 is suitable for the further processing steps in the laboratory. Before they are processed, the collected samples do not have to be transferred to another vessel, as is necessary in the currently available equipment.

FIG. 7 shows a flow chart of an illustrative embodiment of a method 700 for obtaining samples from the respiratory air according to one illustrative embodiment. The method 700 can be implemented for example using the attachment device described with reference to FIG. 1 for a sample collection device for obtaining samples from the respiratory air.

The method 700 initially comprises a step 701 in which the exhaled respiratory air is guided into a centrifugal separator. This is optionally assisted by a pump. Finally, in a step 703, the samples from the respiratory air are collected at the wall of the centrifugal separator utilizing the centrifugal force.

The method can comprise a further step in which the collector is separated. Here, the attachment device and the sample collection device coupled mechanically to the attachment device are separated. The sample collection device with the collected sample is then ready for further analysis. The attachment device can be discarded, or it can be connected again to a sample collection device in order to collect a further sample.

If an illustrative embodiment comprises an “and/or” link between a first feature and a second feature, this should be interpreted as meaning that the illustrative embodiment has both the first feature and the second feature in accordance with one embodiment and either only the first feature or only the second feature in accordance with a further embodiment.

1. An attachment device for a sample collection device configured for obtaining samples from exhaled respiratory air, the attachment device comprising:
an inlet tube;
a dip tube configured to discharge the respiratory air from the sample collection device;
an outlet opening configured to conduct the respiratory air to the sample collection device; and
at least a portion of a spiral path, the portion running into the outlet opening.

2. The attachment device as claimed in claim 1, wherein the inlet tube has an interface for fastening a mouthpiece.

3. The attachment device as claimed in claim 1, wherein the inlet tube is configured as a mouthpiece.

4. The attachment device as claimed in claim 1, wherein the dip tube has a protrusion that protrudes beyond a stop of the attachment device for the sample collection device.

5. The attachment device as claimed in claim 1, further comprising:
an interface configured for mechanical, reversible connection of the attachment device and the sample collection device.

6. A collector comprising:
a sample collection device; and
an attachment device coupled mechanically to the sample collection device, the attachment device comprising:
an inlet tube;
a dip tube configured to discharge respiratory air from the sample collection device;
an outlet opening configured to conduct the respiratory air to the sample collection device; and
at least a portion of a spiral path, the portion running into the outlet opening.

7. The collector as claimed in claim 6, wherein the attachment device tapers at a side directed toward the sample collection device and has an opening, and wherein the attachment device is placed or placeable directly onto the sample collection device and is suitable for further processing steps in a laboratory.

8. The collector as claimed in claim 6, wherein the sample collection device is designed as a commercially available reaction vessel.

9. The collector as claimed in claim 6, wherein the sample collection device is configured as a reaction vessel with a volume of between 0.05 ml and 3 ml.

10. The collector as claimed in claim 6, wherein the sample collection device is configured as a centrifuge tube with a volume of between 2.5 ml and 25 ml.

11. A method for obtaining samples from exhaled air, the method comprising:
guiding the exhaled air into a centrifugal separator; and
collecting the samples from the exhaled air at a wall of the centrifugal separator using centrifugal force.

12. The method as claimed in claim 11, further comprising:
separating a sample collection vessel of the centrifugal separator from an attachment device of the centrifugal separator.

13. The method as claimed in claim 11, wherein the guiding of the exhaled air into the centrifugal separator is assisted by a pump.

14. (canceled)
